SEQUENCE LISTING (5 sequences)

SEQ ID NO: 1 - DNA, 21 bp, Artificial Sequence
  Synthetic
aacagggactcagggaccaga

SEQ ID NO: 2 - DNA, 21 bp, Artificial Sequence
  Synthetic
aaatgaatgttcctggggcgc

SEQ ID NO: 3 - DNA, 21 bp, Artificial Sequence
  Synthetic
aagagaaatcgaaaccgaaaa

SEQ ID NO: 4 - DNA, 21 bp, Artificial Sequence
  Synthetic
aagaacccaattaagcgcaag

SEQ ID NO: 5 - protein, 14 aa, Mus musculus
  MISC_FEATURE (6)..(6): Modified by nitration
SSVSDYGNYDIIVR

(19) United States
(12) Patent Application Publication
(10) Pub. No.: US 2014/0323545 A1
(43) Pub. Date: Oct. 30, 2014

(54) NITRATED SPHINGOSINE 1-PHOSPHATE 3 RECEPTOR AS A PREDICTOR OF ACUTE LUNG INJURY-ASSOCIATED MORTALITY

(71) Applicant: Joe G.N. Garcia, Tucson, AZ (US)

(72) Inventor: Joe G.N. Garcia, Tucson, AZ (US)

(21) Appl. No.: 14/236,901

(22) PCT Filed: Aug. 6, 2012
(86) PCT No.: PCT/US2012/049761

§ 371 Date: Jun. 10, 2014

Related U.S. Application Data
(60) Provisional application No. 61/515,527, filed on Aug. 5, 2011.

(51) Int. Cl. G01N 33/53

(52) U.S. Cl. CPC G01N 33/53
USPC 514/44 A; 435/7.92; 436/501

(57) ABSTRACT

The disclosure relates to a method of determining risk of mortality from Acute Lung Injury (ALI), sepsis, or a combination thereof in a patient, as well as a method of diagnosing ALI in a patient with sepsis based on the presence of tyrosine-nitrated sphingosine 1-phosphate 3 receptor (S1P3R) protein. The disclosure additionally relates to a method of treating an Acute Lung Injury (ALI) patient with sepsis based on the presence of tyrosine-nitrated S1P3R protein.

This application claims the benefit of priority to U.S. Provisional Patent Application Ser. No. 61/515,527, filed Aug. 5, 2011, and is incorporated by reference herein in its entirety.

### BACKGROUND OF THE INVENTION

Acute lung injury (ALI) is characterized by profound inflammation, increased vascular permeability, and alveolar flooding. This combination of symptoms frequently results in acute respiratory failure. The incidence of ALI in the United States is higher than in other developed countries, and even though the ALI mortality rate has improved over the past four decades it remains at a relatively high 30-40% (Spragg et al., Am J Respir Crit Care Med (2010) 181:1121-27; Frutos-Vivar et al., Curr Opin Crit Care (2004) 10:1-6; MacCallum & Evans, Curr Opin Crit Care (2005) 11:43-49; Erickson et al., Crit Care Med (2009) 37:1574-79; Phua et al., Am J Respir Crit Care Med (2009) 179:220-27; Vadasz & Sznajder, Crit Care Med (2010) 183:1147-52). One impediment to improving ALI mortality rates is the paucity of reliable biomarkers for diagnosis, prognosis and responses to therapy. Identifying ALI biomarkers would permit improved clinical decision-making, better informed family discussions, and better use of healthcare resources (Levitt et al., J Intensive Care Med. (2009) 24:151-67). Biomarkers can be important for early detection of lung injury, as well as risk stratification for clinical trials, and, ultimately, tailoring specific therapies to individual patients.

There has been a growing interest in the field for ALI biomarkers that have recognized roles in vascular homeostasis including inflammatory factors, such as IL-1β, IL-6, IL-8 and TNFα (Meduri et al., Chest (1995) 107:1062-73; Miller et al., Crit Care Med (1996) 24:1448-54; Geiser et al., Am J Respir Crit Care Med (2001) 163:1384-88; Tremblay et al., Crit Care Med (2002) 30:1693-1700; Rubin et al., J Clin Invest (1990) 86:474-80), coagulation factors such as protein C and thrombomodulin (Ware et al., Am J Physiol Lung Cell Mol Physiol (2003) 285:L514-21), and endothelial cell-derived factors such as vWF (Rubin et al., J Clin Invest (1990) 86:474-80, Ware et al., Crit Care Med (2001) 29:2325-31), VEGF (Thickett et al., Am J Respir Crit Care Med (2002) 166:1332-37; Abadie et al., Eur Respir J (2005) 25:139-46; Becker et al., Am J Physiol Lung Cell Mol Physiol (2001) 281:L 1500-11), angiopoietin-2 (van der Heijden et al., Thorax (2008) 63:903-09; Parikh et al., PLoS Med (2006) 3:e46), ICAM-1 (Flori et al., Pediatr Crit Care Med (2003) 4:315-21; Agouridakis et al., Respir Res (2002) 3:25) and E- or P-selectin (Donnelly et al., Lancet (1994) 344:215-19; Okajima et al., Transl Res (2006) 148:295-300; Sakamaki et al., Am J Respir Crit Care Med (1995) 151:1821-26). This focus is supported by the presence of marked disruption of vascular integrity and increased vascular permeability, reflecting actions of bioactive agonists, cellular components and mechanical stresses on lung vascular integrity, that are cardinal features of inflammatory lung injuries such as ALI (Sakamaki et al., Am J Respir Crit Care Med (1995) 151:1821-26; Garcia et al., Am J Respir Crit Care Med (2011) 183:1287-89). Plasma levels of IL-6 and IL-8 are well-understood ALI biomarkers, characterized by elevated levels in spontaneously ventilating patients with ALI prior to endotracheal intubation (Cepkova et al., Crit Care (2006) 10:RI26). Such elevated levels are also associated with ALI morbidity and mortality (Parsons et al., Crit Care Med (2005) 33:1-6; discussion 230-232). Specifically, IL-6 haplotypes are associated with ALI susceptibility (Flores et al., Transl Res (2008) 152:11-17) and increased organ dysfunction and mortality in critically ill patients (Sutherland et al., Arch Intern Med (2005) 165:75-82). However, the correlation between the majority of biochemical markers, pathophysiologic variables and clinical outcomes remains nonspecific and of uncertain clinical value (Cribbs et al., Crit Care Med (2008) 36:2457-59).

Another characteristic of ALI is excessive production of reactive oxygen and nitrogen species. Analytical advancements now allow nitration of tyrosine residues to 3-nitrotyrosine to be identified as selective modifications derived from the formation of nitric oxide-derived oxidants (Nicholls et al., Circulation (2007) 116:2315-24; Parastatidis et al., J Biol Chem (2008) 283:33846-53; Parastatidis et al., Circ Res 2007; 101:368-76; Shishehbor et al., JAMA (2003) 289:1675-80). Quantification of 3-nitrotyrosine protein nitration and/or the resulting compromise in biological activities has the potential to deliver specific and clinically relevant biomarkers (Parastatidis et al., J Biol Chem (2008) 283:33846-53; Parastatidis et al., Circ Res 2007; 101:368-76; Shishehbor et al., JAMA (2003) 289:1675-80; Lanone et al., Biochem J (2002) 366:399-404; Zheng et al., J Clin Invest (2004) 114:529-41; Thomson et al., Am J Respir Cell Mol Biol (2007) 36:152-57). Cerruloplasmin, transferrin, α1-protease inhibitor, α1-anti-chymotrypsin and β-chain fibrinogen are all nitrated in ALI (Gole et al., Am J Physiol Lung Cell Mol Physiol (2000) 278:L961-67) with several additional proteins implicated in mouse sepsis models (α2-macroglobulin, apolipoprotein A-1, haptoglobin, Vitamin D-binding protein) previously observed to undergo nitration (Ghesquiere et al., Mol Cell Proteomics (2009) 8:2642-52). Several biomarkers exhibit genetic variations associated with ALI susceptibility as well as outcome (Gao et al., Am J Respir Cell Mol Biol (2006) 34:487-95; Zhai et al., Thorax (2007) 62:718-22) and are known to influence vascular barrier function and the increased vascular permeability characteristic of ALI and other inflammatory lung injuries. For example, VEGF increases vascular permeability in vitro (Becker et al., Am J Physiol Lung Cell Mol Physiol (2001) 281:L1500-11; Behzadian et al., FASEB J(2003) 17:752-54; Chang et al., Microvasc Res (2000) 59:265-77) and in vivo (Fu, et al., 2004, Microvasc Res, 68:51-62; Fu, et al., 2003, Am J Physiol Heart Circ Physiol, 284:H2124-213, Bates, et al., 2001, J Physiol. 533:263-272), involving VEGFR2 activation, calcium influx, activation of PLCγ, Rho-Rac and junctional signaling (Bates, Cardiovasc Res (2010) 87:262-71). Angiopoietin-2 (ang-2) is packaged in secretory Weibel-Palade bodies, and simultaneously released from endothelial cells (EC) upon injury (Fiedler, Trends Immunol (2006) 27:552-58), producing disruption of cell-cell adhesion linkages and reduced vascular integrity and edema (Fiedler, Trends Immunol (2006) 27:552-58; Roviezzo et al., J Pharmacol Exp Ther (2005) 314:738-44). Excess circulating ang-2 may contribute to pulmonary vascular leak in sepsis in humans (Parikh et al., PLoS Med (2006) 3:e46), related to pulmonary dysfunction and ICU mortality throughout the course of septic shock (van der Heijden et al., Intensive Care Med (2009) 35:1567-1574). The search for reliable biomarkers, however is somewhat hindered by the inherent heterogeneity of the disease along with the consistent lack of correlation between biochemical markers, pathophysiologic variables and clinical outcomes (Cribbs et al., Crit Care Med (2008) 36:2457-59).

Thus, there remains a need in the art for more relevant and reliable biomarkers for diagnosis of ALI and the risk of morbidity and mortality in ALI patients.

### SUMMARY OF THE INVENTION

This invention as disclosed provides methods for determining risk of mortality from Acute Lung Injury (ALI), sepsis, or a combination thereof in a patient, comprising: (a) obtaining a blood plasma sample from the patient; and (b) performing an assay to measure tyrosine-nitrated sphingosine 1-phosphate 3 receptor (S1P3R) protein concentration in the plasma sample taken from the patient, wherein there is an increased risk of mortality when said S1P3R protein concentration is 200 pg/mL or higher. In certain embodiments there is an increased risk of mortality when the tyrosine-nitrated S1PR3 concentration is 250 pg/mL or higher. In one embodiment, the tyrosine-nitrated S1P3R protein is isolated from a vascular endothelial cell-derived microparticle in said patient blood plasma sample. In another embodiment, the tyrosine-nitrated S1P3R protein concentration is measured by an antibody-based detection method. In a specific embodiment, the antibody-based detection method is for example ELISA or western blot. In certain embodiments, the risk of mortality is increased by 10%, 20%, 30%, 40%, or 50%.

This invention as disclosed also provides methods for diagnosing ALI in a patient with sepsis, the steps comprising. (a) obtaining a blood plasma sample from the patient; (b) performing an assay to measure tyrosine-nitrated S1P3R protein concentration in the plasma sample taken from the patient, (c) performing an assay to measure tyrosine-nitrated S1P3R protein concentration in the plasma sample taken from a control patient, and (d) comparing the protein concentrations determined in steps (b) and (c); wherein ALI is diagnosed when S1P3R protein concentration is higher in the plasma sample determined in step (b) than in the plasma sample determined in (c). In one embodiment, the tyrosine-nitrated S1P3R protein is isolated from a vascular endothelial cell-derived microparticle in said patient blood plasma sample. In another embodiment, the tyrosine-nitrated S1P3R protein concentration is measured by an antibody-based detection method. In a specific embodiment, the antibody-based detection method is for example ELISA or western blot.

This invention as disclosed herein also provides methods for treating an Acute Lung Injury (ALI) patient with sepsis, comprising administering aggressive ALI treatment when S1P3R concentrations in the patient's plasma are at least 200 pg/mL. In a certain embodiment, aggressive ALI treatment is given to the patient when S1P3R concentrations in the patient's plasma are at least 250 pg/mL.

The invention as disclosed presents certain advantages over what is known in the art. The inventor discloses herein for the first time that S1PR3 can be used as biomarker for ALI. The embodiments disclosed herein fill the need in the art for more relevant and reliable biomarkers for diagnosis of ALI and the risk of morbidity and mortality in ALI patients. An additional advantage provided herein is the availability of antibodies for use in the disclosed methods. Another advantage provided herein is that the methods disclosed herein are plasma based rather that biopsy or symptomology based, which allows for rapid continuous monitoring from plasma. One further advantage is that the methods disclosed herein are reactive, i.e., allowing for continuous monitoring of nitrated S1PR3 levels with treatment to assess success of the treatment.

Additional features and advantages are described herein, and will be apparent from the following Detailed Description, Drawings and the claims.

### BRIEF DESCRIPTION OF THE DRAWINGS

FIG. 1 shows that plasma nitrated S1PR3 protein and mRNA levels are increased in murine models of LPS-induced ALI, wherein FIGS. 1A and 1C are graphs showing absolute (FIG. 1A) and relative (FIG. 1C) amounts of S1PR3 protein in control and lipopolysaccharide (LPS)-challenged mice; FIGS. 1B and 1D are photographs of immunoblotting experiments showing increased S1PR3 in plasma (FIG. 1B) and lung tissue (FIG. 1D) in mice challenged with LPS, and further the presence of nitrotyrosine residues in said S1PR3 markers; and FIG. 1E is a photograph showing results of immunoblot experiments showing increase in plasma S1PR3 in mice challenged with LPS, LPS+high molecular weight hyaluronan (HMWHA) or LPS+hepatocyte growth factor (HGF).

FIG. 2A is a graph showing that intratracheal S1PR3 siRNA administration significantly decreased lung S1PR3 expression; the inset shows the results of immunoblotting experiments. FIG. 2B is a graph showing that intratracheal S1PR3 siRNA administration significantly decreased attenuated mechanical ventilation-induced leukocyte bronchioalveolar lavage (BAL) infiltration. FIG. 2C is a graph showing that intratracheal S1PR3 siRNA administration significantly decreased vascular permeability as detected by tissue albumin levels.

FIG. 3A is a graph showing increased plasma levels of S1PR3 in human sepsis-induced and trauma-induced ALI patients. FIG. 3B is a photograph of immunoblotting experiments showing increased tyrosine-nitrated S1PR3 levels in human sepsis-induced and trauma-induced ALI patients. FIGS. 3C and 3D are graphs showing that increased tyrosine-nitrated S1PR3 levels in human sepsis-induced and trauma-induced ALI patients are associated with increased risk of sepsis/ALI mortality.

FIGS. 4A through 4D are photographs of immunoblotting experiments showing that human endothelial cell S1PR3 receptor exhibits basal tyrosine nitration and release that is substantially increased by SIN-1 or LPS challenge.

FIGS. 5A through 5C are photographs of immunoblotting experiments showing that tyrosine-nitrated S1PR3 is released from human lung endothelial cells following exposure to vascular barrier-disruptive agents or increased mechanical stress.

FIG. 6A is a photograph of immunoblotting experiments and FIGS. 6B and 6C are graphs showing that tyrosine-nitrated S1PR3 released from microparticles contributes to endothelial cell barrier disruption. FIG. 6B shows transendothelial electrical resistance (TER) measurements in response to addition to S1PR3-containing collected microparticles demonstrated dose-dependent reductions in EC barrier integrity. FIG. 6C shows microparticles generated from EC with prior reductions in S1PR3 expression via incubation with 3 μM of S1PR3 siRNA (inset), demonstrating reduced capacity of the microparticles to decrease normalized resistance.

### DETAILED DESCRIPTION

Disclosed herein is a novel ALI biomarker, S1PR3 identified by searching for nitrated plasma proteins in murine ALI models. This novel plasma ALI biomarker S1PR3 (aka endothelial differentiation gene 3 (Edg3)) is a critical G-protein coupled receptor for the angiogenic factor, SIT (Garcia et al., J Clin Invest (2001) 108:689-701; English et al., FASEB J (2000) 14:2255-65), and a direct participant in regulation of vascular permeability (Sammani et al., Am J Respir Cell Mol Biol (2010) 43:394-402; Shikata et al., FASEB J (2003) 17:2240-49). As shown herein, S1PR3 is minimally detectable in the circulation of unchallenged mice but significantly released following a variety of inflammatory and vascular barrier-disruptive factors (LPS and excessive mechanical ventilatory stress), and rapidly released in vitro into the extracellular media by LPS, thrombin, low molecular weight hyaluronan (LMHA) and excessive cyclic stretch. As further shown herein, the clinical relevance of these findings was confirmed by increased detection of S1PR3 in plasma of humans with either sepsis, trauma-induced ALI or sepsis-induced ALI (compared to trauma and cases without ALI or sepsis). Of note, elevated plasma S1PR3 levels were significantly associated with increased mortality in both sepsis and ALI cases. These finding suggested that S1PR3 can be an informative early prognostic biomarker of vascular injury in ALI.

In addition to S1PR3, sphingosine 1-phosphate (S1P) binds to other plasma membrane heptahelical SIT receptors 1 (Edg1), 2 (Edg5), 4 (Edg6) and 5 (Edg8) expressed in a variety of cell types including endothelium (Garcia et al., J Clin Invest (2001) 108:689-701; Spiegel & Milstien, Nat Rev Mol Cell Biol (2003) 4:397-403; Pyne & Pyne, Pharmacol Ther (2000) 88:115-131). Human endothelial cells (EC) exhibit elevated expression of S1PR1 and S1PR3 with S1PR1 signaling coupled to the Gᵢ pathway and Rac1 activation whereas S1PR3 signaling couples to the Gᵢ, G_{q/11} and G_{12/13} pathways and activates RhoA to a much greater extent than Rac1 (Garcia et al., J Clin Invest (2001) 108:689-701; Waeber et al., Drug News Perspect (2004) 17:365-82). Activated RhoA binds to and activates the serine/threonine kinase, ROCK (Sahai et al., EMBO J (1998) 17:1350-61; Fujisawa et al., J Immunol (1998) 161:5627-32), involved in EC barrier disruption pathways (Singleton et al., J Biol Chem (2006) 281:34381-93; Tasaka et al., Am J Respir Cell Mol Biol (2005) 32:504-10). Previously disclosed experimental evidence indicated that the S1PR1 receptor is needed for endothelial barrier enhancement, whereas S1PR3 is needed for EC barrier disruption in vitro and in vivo (Singleton et al., J Biol Chem (2006) 281:34381-93; Singleton et al., FASEB J (2005) 19:1646-56, Sun et al., J Allergy Clin Immunol 126:241-49) but any potential for using S1PR3 as a biomarker for ALI was unappreciated.

More recently, posttranslational modification of circulating plasma proteins has been recognized as a potential biomarker in inflammatory disorders that can reflect disease severity and progression (Kooy et al., Crit Care Med (1997) 25:812-19; Sittipunt et al., Am J Respir Crit Care Med (2001) 163:503-10; Lange et al., Crit Care (2010) 14:RI29). In addition to post translational modifications of S1PR3 phosphorylation on tyrosine and threonine residues, disclosed herein is the heretofore undisclosed observation that S1PR3 is nitrated at one or more tyrosine residues and released within EC microparticles following challenge with agents that induce lung vascular barrier disruption. Endothelial microparticles are complex vesicular structures shed by activated or apoptotic EC, and contain enzymes, transcription factors and mRNA. Endothelial cells release microparticles after activation by a variety of inflammatory stimuli, such as TNF-α (Combes et al., J Clin Invest (1999) 104:93-102) and other inflammatory cytokines (Szotowski et al., Cardiovasc Res (2007) 73:806-12), lipopolysaccharide (LPS), reactive oxygen species (Szotowski et al., Cardiovasc Res (2007) 73:806-12), plasminogen activator inhibitor (Brodsky et al., Circulation (2002) 106:2372-78), thrombin (Sapet et al, Blood (2006) 108:1868-76), camptothecin (Simak et al., Transfusion (2002) 42:334-42), C-reactive protein (Wang et al., J Vasc Res (2007) 44:241-48) and uremic toxins (Faure et al., Thromb Haemost (2006) 4:566-73). Low shear stress is associated with in vivo endothelial microparticle release in end-stage renal disease (Boulanger et al., Hypertension (2007) 49:902-08) and sepsis autopsy specimens demonstrated intense nitrotyrosine immunoreactivity in the endocardium, myocardium, and coronary vascular endothelium (Kooy et al., Grit Care Med (1997) 25:812-19).

As further disclose herein, increased circulating levels of nitrated S1PR3 were found to be present in plasma from mice and humans with ALI. EC barrier-disruptive agents known to increase reactive oxygen species (ROS) and peroxynitrite production induced specific post-translational modifications of plasma membrane S1PR3 (tyrosine and threonine phosphorylation) as well as tyrosine nitration. In contrast, only S1PR3 nitration and not phosphorylation appeared to occur in released microparticles. Nitrotyrosine was detectable in BAL fluid samples for up to 14 days after ALI onset, but not in BAL of healthy volunteers (Sittipunt et al., Am J Respir Grit Care Med (2001) 163:503-10).

The utility of nitrated S1PR3 receptor as a novel ALI biomarker was confirmed in murine ALI and human ALI samples, and increased S1PR3 levels were significantly associated with worsened outcome in critical care sepsis patients. Several EC barrier-disruptive agents, including lipopolysaccharide (LPS) and thrombin-induced S1PR3 nitration and release into 100 to 1000 nm microparticle vesicles, significantly increased EC permeability.

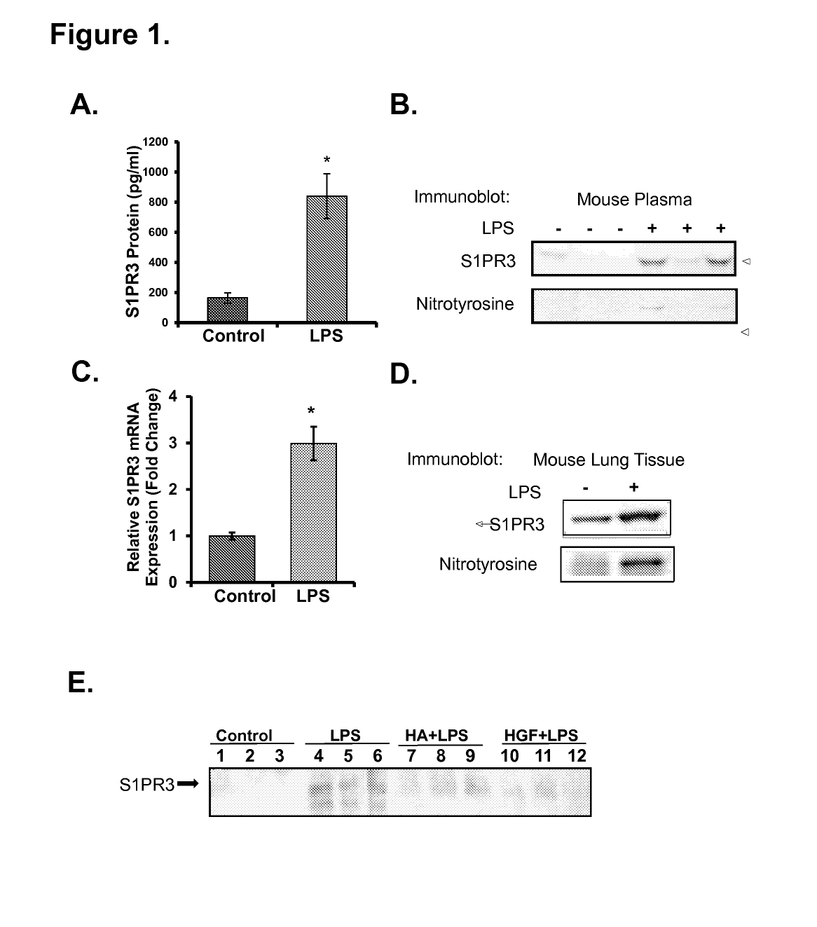

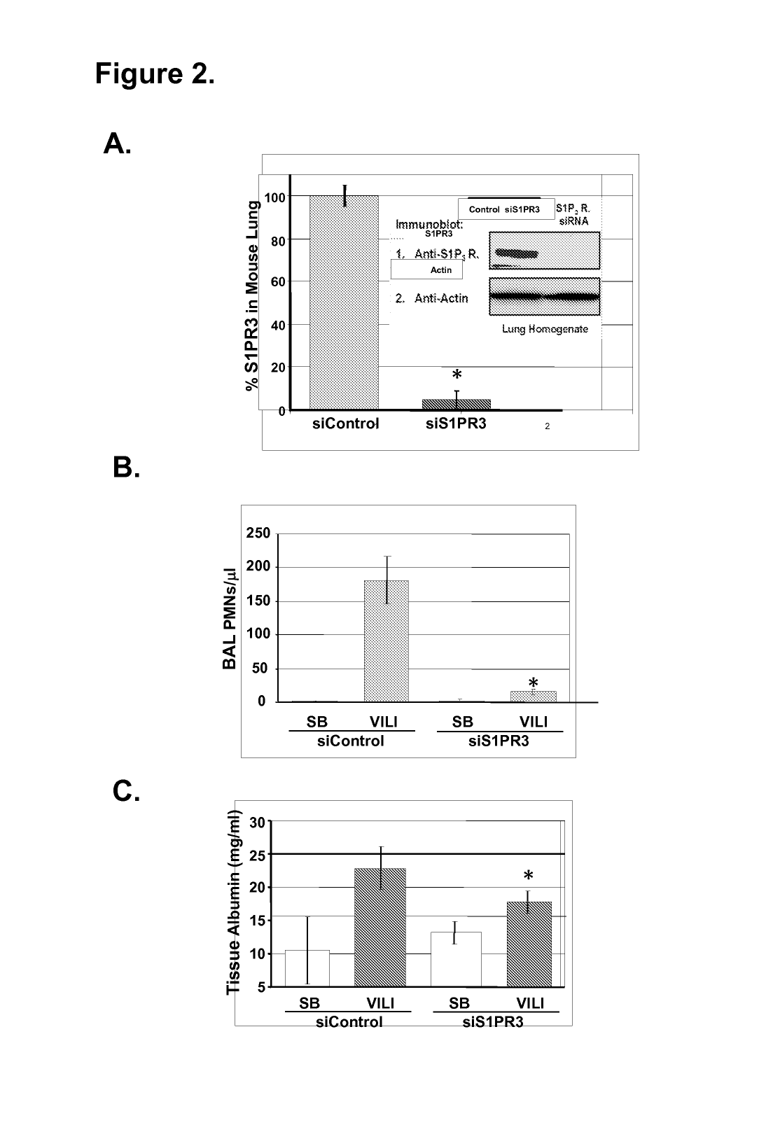

The disclosure herein demonstrated that EC injury-producing agents, possibly through inflammatory factors TNFα and thrombin (Tang et al., PNAS (2006) 103:13777-82), induced EC S1PR3 nitration and shedding within microparticles. Further, the disclosure demonstrated that nitrated S1PR3-containing microparticles enhanced EC barrier-disruption in vitro, consistent with microparticle effects in sickle cell disease (Shet et al., Blood (2003) 102:2678-83) which suggest microparticles as a measure of EC injury as well as a cellular source of vascular dysfunction-inciting agents. LPS, in combination with cytokines, has been noted to increase EC microparticle production (Aird et al., Blood (2003) 101:3765-77) with detection in the plasma of healthy subjects and increased levels under pathological conditions associated with increased thrombotic risk and endothelial dysfunction (Leroyer et al., Thromb Haemost (2010) 104:456-63). Specifically, shown herein are results demonstrating that circulating S1PR3 is increased in the circulation of mice with ALI (see FIGS. 1A & 1C) and decreasing S1PR3 is associated with decreased vascular hyper-permeability in vivo (see FIGS. 2B & 2C). Utilizing both in vitro and in vivo models of lung injury and pulmonary vascular permeability, it is shown herein that S1PR3 was tyrosine nitrated and released in microparticles following exposure to agents that induce lung injury and vascular barrier disruption. In addition, significantly increased circulating plasma levels of nitrated S1PR3 receptor was observed in mice with ALI and humans with ALI, with increased levels conferring risk for sepsis and ALI mortality. These results confirmed that nitrated S1PR3 is a useful biomarker for sepsis and ALI.

In the practice of the invention disclosed herein, conventional and standard techniques may be used for recombinant DNA molecule, protein, and antibody production, as well as for tissue culture and cell transformation. Enzymatic reactions and purification techniques are typically performed according to the manufacturer's specifications or as commonly accomplished in the art using conventional procedures known in the art, or as described herein. Unless specific definitions are provided, the nomenclature utilized in connection with, and the laboratory procedures and techniques of analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

Further, the terminology used herein is for the purpose of exemplifying particular embodiments only and is not intended to limit the scope of the invention as disclosed herein. Any method and material similar or equivalent to those described herein can be used in the practice of the invention as disclosed herein and only exemplary methods, devices, and materials are described herein.

All patents and publications mentioned herein are incorporated by reference in their entirety for the purpose of describing and disclosing the proteins, enzymes, antibodies, vectors, host cells, reagents and methodologies reported therein that might be used with and in the invention as disclosed herein. However, nothing herein is to be construed as an admission that the invention is not entitled to antedate such disclosure by virtue of prior invention.

As utilized in accordance with the present disclosure, the following terms unless otherwise indicated, shall be understood to have the meanings disclosed herein.

The term “biological sample” as used herein includes, but is not limited to a tissue or bodily fluid obtained from an animal, preferably a mammal and most preferably a human. For example, a biological sample can be biopsy material, blood, blood plasma, serum or cellular fraction thereof, derived from a biological source. In specific embodiments, a biological sample is alveolar lavage. In one embodiment, the mammal is a human suspected of having or previously diagnosed as having or in need of screening for an inflammatory lung injury, and in certain embodiments the inflammatory lung injury is ALI.

The term “short interfering RNA” or “siRNA” as used herein refers to a double stranded nucleic acid molecule capable of RNA interference or “RNAi.” As used herein, siRNA molecules need not be limited to those molecules containing only RNA, but further encompasses chemically modified nucleotides and non-nucleotides having RNAi capacity or activity.

Provided herein are a methods for determining risk of mortality for an Acute Lung Injury (ALI) patient with sepsis, comprising performing an assay, preferably an immunoassay specific for tyrosine-nitrated sphingosine-1-phosphate 3 receptor (S1PR3) on a blood plasma sample taken from the patient and determining tyrosine-nitrated sphingosine 1-S1P3R protein concentration therein, wherein there is an increased risk of mortality when said S1P3R concentration is 200 pg/mL or higher.

As used herein, “Acute Lung Injury” or “ALI” is a diffuse heterogeneous lung injury characterized by hypoxemia, non-cardiogenic pulmonary edema, low lung compliance and widespread capillary leakage. ALI is caused by any stimulus of local or systemic inflammation, principally sepsis.

As used herein, the term “risk of mortality” means the likelihood measured in percentages that a patient will die from symptoms associated with ALI and sepsis. In certain embodiments, this risk is increased by 10%, 20%, 30%, 40%, or 50%.

As used herein, the term “tyrosine-nitrated sphingosine 1-phosphate 3 receptor” or “tyrosine-nitrated S1PR3” means S1PR3 that has 3-nitrotyrosine nitration of tyrosine residues as selective modifications derived from the formation of nitric oxide-derived oxidants (Nicholls et al., Parastatidis et al., Parastatidis et al., Shishehbor et al). In certain embodiments, the concentration of nitrated S1PR3 is measured in any units generally accepted in the art. In a particular embodiment, the concentration is measured in pg/mL. In one specific embodiment, there is an increased risk of mortality when said S1PR3 concentration is 200 pg/mL or higher. In another embodiment, there is an increased risk of mortality when said S1PR3 concentration is 250 pg/mL or higher. In certain embodiments, S1PR3 concentration is measured by any “antibody-based detection method” known to the skilled artisan. In a specific embodiments, the tyrosine nitrated S1P3R protein concentration is measured by ELISA. In yet other embodiments, the tyrosine nitrated S1P3R protein is measured by western blot.

In one embodiment, the tyrosine nitrated S1P3R protein is isolated from a lipopolysaccharide (LPS) microparticle in a blood plasma sample from a patient. As used herein, “LPS microparticles” or “endothelial microparticles” are complex vesicular structures shed by activated or apoptotic endothelial cells, and contain enzymes, transcription factors and mRNA.

Provided herein also are methods for diagnosing ALI in a patient with sepsis, the steps comprising (a) obtaining a blood plasma sample from the patient; (b) performing an assay to measure tyrosine-nitrated S1P3R protein concentration in the plasma sample taken from the patient, (c) performing an assay to measure tyrosine-nitrated S1P3R protein concentration in the plasma sample taken from a control patient, and (d) comparing the protein concentrations determined in steps (b) and (c); wherein ALI is diagnosed when S1P3R protein concentration is higher in the plasma sample determined in step (b) than in the plasma sample determined in (c). As used herein, “control patient” is a patient admitted to the intensive care unit (ICU) but that does not have sepsis or sepsis accompanied by ALI. In one embodiment, the tyrosine nitrated S1P3R protein is isolated from a lipopolysaccharide (LPS) microparticle in a blood plasma sample from a patient. In certain embodiments, S1PR3 concentration is measured by any “antibody-based detection method” known to the skilled artisan. In a specific embodiments, the tyrosine nitrated S1P3R protein concentration is measured by ELISA. In yet other embodiments, the tyrosine nitrated S1P3R protein is measured by western blot.

Provided herein is also a method of treating an Acute Lung Injury (ALI) patient with sepsis, the method comprising administering aggressive ALI treatment to the patient when S1P3R concentrations in the patient's plasma are at least 200 pg/mL. As used herein, “aggressive ALI treatment” is increased monitoring of the patient, giving fluid management to the patient to maintain blood pressure and prevent dehydration, as well as use of broad antibiotic therapy. In a certain embodiment, aggressive ALI treatment is used when the S1PR3 concentration is at least 250 pg/mL.

### EXAMPLES

The invention now will be exemplified for the benefit of the artisan by the following non-limiting examples that depict some of the embodiments by and in which the invention can be practiced.

### Materials and Methods

### 1. Cell Culture and Reagents

Human pulmonary artery (HPAEC) and lung microvascular EC (HLMVEC) were obtained from Cambrex (Walkersville, Md.) and cultured as previously described (Garcia et al., J Clin Invest (2001) 108:689-701) in EBM-2 complete medium (Cambrex) at 37° C. in a humidified atmosphere of 5% CO₂, 95% air, with passages 6-10 used for experimentation. Unless otherwise specified, reagents were obtained from Sigma Chemical Co. (St. Louis, Mo.). Reagents for SDS-PAGE electrophoresis were purchased from Bio-Rad (Richmond, Calif.), Immobilon-P transfer membrane and mouse anti-nitrotyrosine (clone 1A6) antibody from Millipore (Millipore Corp., Bedford, Mass.), and gold microelectrodes from Applied Biophysics (Troy, N.Y.). Rabbit and mouse anti-S1PR3 receptor antibodies were purchased from Exalpha Biologicals (Watertown, Mass.). Rabbit anti-phosphoserine and rabbit anti-phosphothreonine antibodies were purchased from Zymed Laboratories, Inc. (South San Francisco, Calif.). Mouse anti-β-actin antibody and LPS were purchased from Sigma (St. Louis, Mo.). Secondary horseradish peroxidase (HRP)-labeled antibodies were purchased from Amersham Biosciences (Piscataway, N.J.).

### 2. Animal Preparation and Experimental Intervention

Male C57BLl6J mice (8-10 weeks, Jackson Laboratories, Bar Harbor, Me.) were anesthetized with intraperitoneal ketamine (150 mg/kg) and acetylpromazine (15 mg/kg). In LPS-induced ALI models, LPS (2.5 mg/kg) or water (control) was instilled intratracheally. High molecular weight hyaluronan (HMWHA; 1.5 mg/kg), hepatocyte growth factor (HGF; 50 μg/kg) or saline (control) were administered intravenously 4 hrs after LPS challenge, as previously described (Singleton et al., Am J Respir Cell Mol Biol (2007) 37:222-31). Animals were allowed to recover for 24 hours after challenge. Plasma and bronchoalveolar lavage fluid were subsequently collected (LPS, n=10; PBS control, n=8) for S1PR3 receptor nitration analysis and protein/albumin measurement. A ventilator-induced lung injury (VILI) model was designed to produce acute lung injury (tidal volume, 40 ml/kg, 4 hours). In specific experiments, S1PR3 siRNAs or siRNA control (Dharmacon) (10 mg/kg mouse, i.v.) was administered in vivo as previously described (Hong et al., Am J Respir Crit Care Med (2008) 178:605-17; Mirzapoiazova et al., Am J Respir Cell Mol Biol (2010) 44:40-52; McVerry et al., Am J Respir Crit Care Med (2004) 170:987-93). Groups included a spontaneously breathing (SB) group with control siRNA, an SB group challenged with S1PR3 siRNA, a high tidal ventilation group (VILI), and a high tidal ventilation group with S1PR3 siRNA challenge (VILI-siS1PR3) (n=4-6 for all groups). Bronchoalveolar lavage fluid was subsequently collected for protein/albumin measurements.

### 3. Immunoprecipitation (IP) and Immunoblotting (IB)

Cellular materials from treated or untreated HLMVEC were incubated with IP buffer (50 mM HEPES (pH 7.5), 150 mM NaCl, 20 mM MgCl₂, 1% Triton X-100, 0.1% SDS, 0.4 mM Na₃H₂VO₄, 40 mM NaF, 50 μM okadaic acid, 0.2 mM phenylmethylsulfonyl fluoride, and Complete™ protease inhibitor tablet. The samples were then immunoprecipitated with anti-S1PR3 receptor-specific IgG followed by SDS-PAGE in 4-15% polyacrylamide gels, transferred onto Immobilon™ membranes, and developed with specific primary and secondary antibodies. Visualization of immunoreactive bands was achieved using enhanced chemiluminescence (ECI) (Amersham Biosciences). Two-dimensional electrophoresis (2-DE) was modified from the protocol described previously (Zhao et al., Cell Signal (2009) 21:1945-60). HLMVECs were treated with LPS (0 to 24 hrs, 1 μg/mL), and then immunoprecipitated with anti-S1PR3. Eluted samples were loaded onto an IPG strip (Amersham Biosciences), which was rehydrated for 12 h followed by isoelectric focusing steps of 500 Vhr, 1000 Vhr, and 8000 Vhr using the IPGphor IEF system (Amersham Biosciences). Second dimension separation was run using an XCell Surelock mini-cell system (Invitrogen) in 4-20% gels. Proteins were then transferred onto Immobilon membranes, and developed with specific primary and secondary antibodies.

### 4. Measurement of Human Plasma S1PR3 via an Enzyme-Linked Immunosorbent Assay (ELISA)

Plasma from patients with severe sepsis-induced ALI (n=23), sepsis without ALI (n=24) and non-sepsis controls (n=19) were collected from the Chicago Consortium for Investigating ICU Genetics (CIICUG) protocol, which was approved by the appropriate institutional review board. Costar EIAIRIA plates were coated at 4° C. overnight with mouse anti-S1PR3 (amino acids 302-379) monoclonal antibodies as capture antibodies. Plates were washed, blocked with Blocking Buffer for 2 hrs, and plasma samples from controls and ALI patients added and incubated for 2 hrs. Rabbit anti-S1PR3 (amino acids 140-170) polyclonal antibodies as detection antibodies were added and incubated for 1 hr, then Alexa Fluor 488 goat anti-rabbit IgG was applied and incubated for 30 min. Final concentrations of S1PR3 were detected by Cytofluor 4000 (Applied Biosystems, Foster City, Calif.), with normalization via concentration-response curves utilizing S1PR3 recombinant protein (Novus). In addition to these studies, a limited number of plasma samples were obtained from trauma patients who developed ALI (n=5) and from trauma subjects without ALI (n=5) and analyzed for S1PR3 receptor nitration.

### 5. RNA Isolation and Microarray Analysis

Total RNA was isolated from whole mouse lungs for expression profiling as described previously (Nonas et al., Am J Physiol Lung Cell Mol Physiol (2007) 293:L292-302) using Affymetrix Mouse 430 2.0 arrays and protocols (Affymetrix, Santa Clara, Calif., USA). Chips were scanned using a GeneChip Scanner 3000 (Affymetrix).

6. Construction and Transfection of siRNA Against S1PR3

siRNA sequence(s) targeting human S1PR3 receptor was generated using mRNA sequences from Gen-Bank™ (gi:38788192). For each mRNA (or scramble), two targets were identified. Specifically:

S1PR3 target sequence 1
(SEQ ID NO: 1)
(5′-AACAGGGACTCAGGGACCAGA-3′),
S1PR3 target sequence 2
(SEQ ID NO: 2)
(5′-AAA TGAA TGTTCCTGGGGCGC-3′),
scramble sequence 1
(SEQ ID NO: 3)
(5′-AAGAGAAA TCGAAACCGAAAA-3′),
and
scrambled sequence 2
(SEQ ID NO: 4)
(5′-AAGAACCCAA TT AAGCGCAAG-3′)

were utilized. Sense and antisense oligonucleotides were purchased from Integrated DNA Technologies (Coralville, Iowa). For construction of siRNA, a transcription-based kit from Ambion was used (Silencer™ siRNA construction kit). Human lung EC were transfected with siRNA using siPORTamine™ as the transfection reagent (Ambion, Tex.) according to the protocol provided by Ambion. Cells (˜40% confluent) were serum-starved for 1 hour followed by incubation with 3 μM (1.5 μM of each siRNA) of target siRNA (or scramble siRNA or no siRNA) for 6 hours in serum-free media. Serum-containing media was then added (10% serum final concentration) for 42 h before biochemical experiments and/or functional assays were conducted.

### 7. Determination of Tyrosine Nitration of S1PR3

Solubilized proteins in IP buffer (see above) were immunoprecipitated with rabbit anti-S1PR3 receptor antibody followed by SDS-PAGE in 4-15% polyacrylamide gels and transfer onto Immobilon™ membranes (Millipore Corp., Bedford, Mass.). After blocking nonspecific sites with 5% bovine serum albumin, the blots were incubated with either mouse anti-S1PR3 antibody or mouse anti-nitrotyrosine antibody followed by incubation with horseradish peroxidase (HRP)-labeled goat anti-rabbit or goat anti-mouse IgG. Visualization of immunoreactive bands was achieved using enhanced chemiluminescence (Amersham Biosciences).

### 8. Microparticle Isolation

Cell culture medium containing S1PR3 transfected EC cells was centrifuged once at 200×g for 10 min to obtain a cell pellet. The resulting supernatant was centrifuged twice at 500×g for 10 min, twice at 1,500×g for 15 min, and once at 10,000×g for 30 min. Small microparticles were then pelleted by centrifugation for 1 h at 70,000×g. Pellets from each centrifugation step were resuspended for applying in TER measurement or S1PR3 western blot analysis.

### 9. Measurement of Transendothelial Electrical Resistance (TER)

EC were grown to confluence in polycarbonate wells containing evaporated gold microelectrodes, and TER measurements performed using an electrical cell-substrate impedance sensing system obtained from Applied Biophysics (Troy, N.Y.) as previously described (Garcia et al., J Clin Invest (2001) 108:689-701). TER values from each microelectrode were pooled at discrete time points and plotted versus time as the mean±S.E.

### 10. Statistical Analysis

Student's t test was used to compare the statistical means of data from two or more different experimental groups. Results are expressed as means±S.D. The significance of difference in survival rate between the high (above the median for the study population) and low S1PR3 concentration groups were measured by log-rank test. Acute Physiology and Chronic Health Evaluation II (APACHE II) is a severity-of-disease classification system. Apache II is applied within 24 hours of admission of a patient to an intensive care unit (ICU): an integer score from 0 to 71 is computed based on several measurements; higher scores correspond to more severe disease and a higher risk of death.

### Example 1

### Plasma Nitrated S1PR3 Levels were Increased in Murine Models of ALI

Global screening of mouse plasma proteins modified by nitration identified the peptide S₁₄SVSDY⁺⁴⁵GNYDIIVR₂₇ (SEQ ID NO: 5) with tyrosine residue in position 19 modified by nitration. In silico analysis revealed this peptide to be within the sequence of the S1PR family of receptor proteins. S1PR family proteins were next evaluated as specific targets for nitration in murine models of ALI and in ALI human subjects. Utilizing a well-characterized model of murine ALI induced by 18 hour exposure to intra-tracheal LPS challenge, significantly increased levels of S1PR3 were detected in plasma from LPS-induced ALI mice compared to control mice (5 fold increase, p<0.01) (FIG. 1A, 1C). Increased murine lung gene and protein expression of S1PR3 was also observed as assessed by Affymetrix microarrays and immunoprecipitation from lung homogenates (FIG. 1B, 1D) with increased levels of S1PR3 tyrosine nitration (FIG. 1C, 1D). Interestingly, the addition of vascular barrier-promoting agonists such as high molecular weight hyaluronan (HMWHA) or HGF (Singleton et al., Am J Respir Cell Mol Biol (2007) 222-31; Singleton et al., J Biol Chem (2006) 281:34381-93; Singleton et al., Am J Physiol Lung Cell Mol Physiol (2010) 299:L639-51; Liu et al., Am J Respir Cell Mol Biol (2001) 24:711-19) significantly attenuated the increase in nitrated S1PR3 receptor recovered in plasma from LPS-challenged ALI mice (FIG. 1E). In addition, reductions in lung S1PR3 expression by intra-tracheal siRNA administration (FIG. 2A) significantly decreased lung S1PR3 expression and attenuated mechanical ventilation-induced leukocyte infiltration and vascular permeability (FIG. 2B, 2C) indicating that S1PR3 contributed to ALI and represented a viable ALI biomarker.

### Example 2

### Plasma Levels of S1PR3 were Increased in Human Sepsis and ALI and Associated with Increased Mortality Risk

Sixty-six critical care (ICU) subjects were investigated for plasma S1PR3 protein concentration. Their clinical characteristics are listed in Table 1.

TABLE 1
Clinical Characteristics of Participants

| | Controls | Sepsis | Sepsis-induced ALI |
|---|---|---|---|
| Male | 9 | 12 | 12 |
| Female | 10 | 12 | 11 |
| African-American | 11 | 12 | 8 |
| European-American | 8 | 11 | 12 |
| Age | 59 ± 17 | 61 ± 16 | 52 ± 19 |
| Apache II Score | 19 ± 6.6 | 28 ± 6.5* | 26 ± 8.9* |
| Mortality Rate (%) | 0 | 25 | 47 |

*p < 0.05 versus Controls

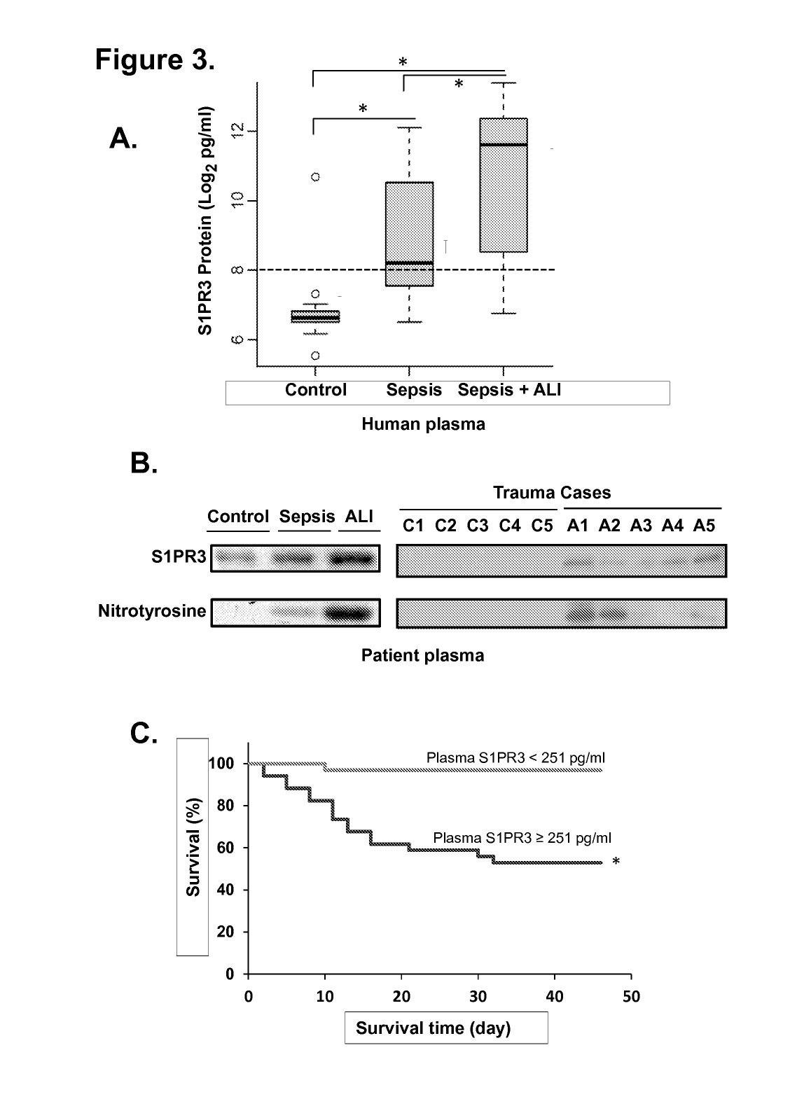

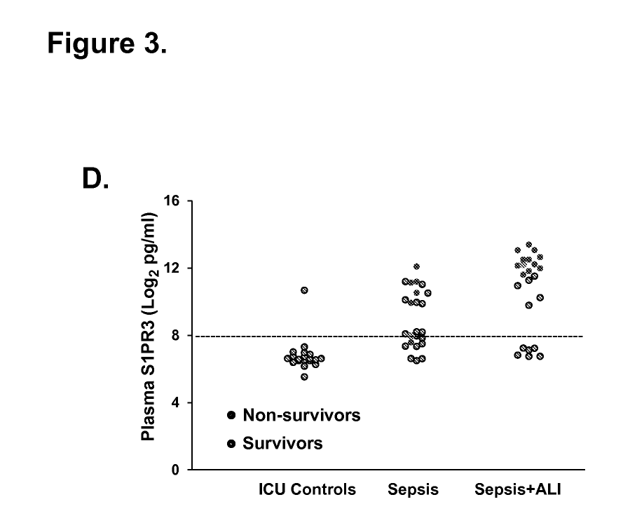

ELISA-detected S1PR3 levels were significantly increased in human plasma from twenty-three patients with sepsis-induced ALI (p<0.01) as well as in twenty-four sepsis patients without ALI (p<0.01) when compared to nineteen ICU control subjects (FIG. 3A). S1PR3 levels were below the median value for the population (251 pg/mL) in the majority of ICU controls (63%, <100 pg/mL) and only one individual (1/19) with an S1PR3 level greater than 251 pg/mL. In contrast, 16 of 24 patients with sepsis and 17 of 23 of patients with ALI exhibited S1PR3 levels greater than 251 pg/mL. In addition, the most significant S1PR3 elevations were detected in severe sepsis-induced ALI cases (3537±664 pg/mL) with significant S1PR3 levels in the sepsis alone cohort (959±225 pg/mL) compared to ICU controls (180±84 pg/mL) (*p<0.01). Plasma protein levels of nitrated S1PR3 from a limited cohort of trauma-induced ALI cases were also significantly increased compared to trauma ICU controls without ALI (FIG. 3B). Importantly, plasma S1PR3 levels >251 pg/mL were significantly associated with increased mortality in both sepsis and ALI ICU cases (p<0.01) (FIGS. 3C and 3D).

### Example 3

### Human Endothelial Cells (EC) Released Nitrated S1PR3 Following NO Donor Exposure

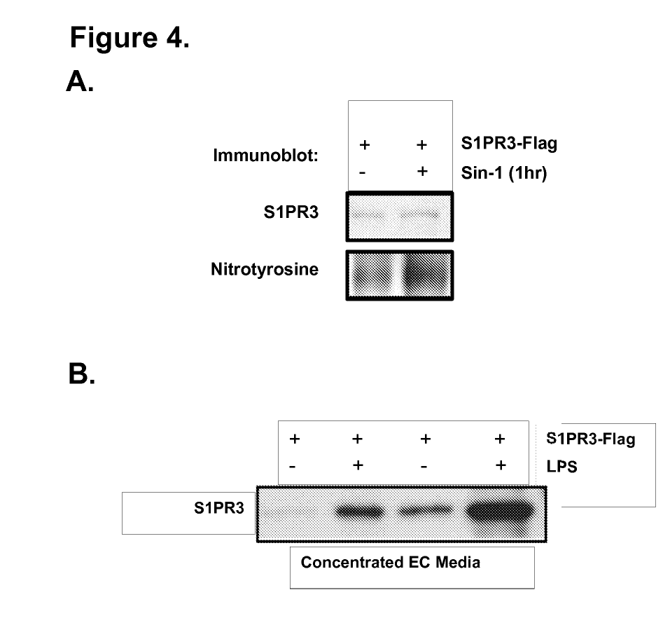

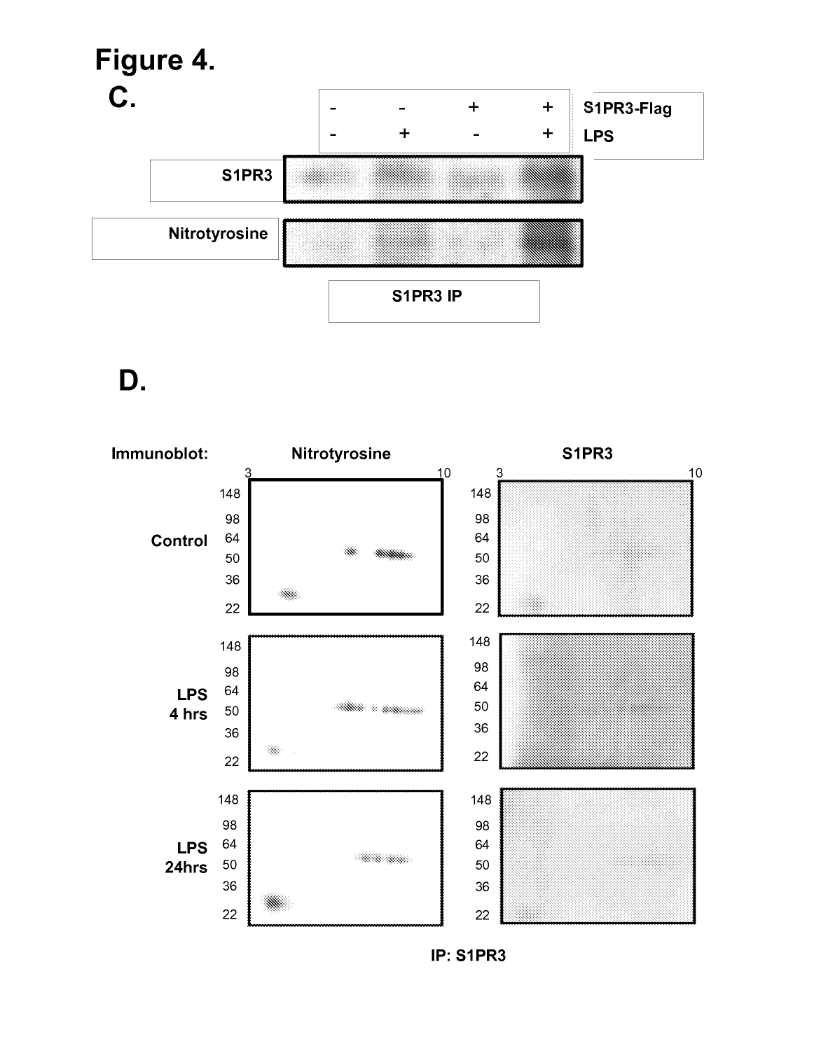

In silico analysis identified 10 potential S1PR3 tyrosine nitration sites including the peptide sequence identified by mass spectroscopy in the original exploratory studies. To confirm that S1PR3 receptor is tyrosine nitrated, Flag-tagged S1PR3 receptor was over-expressed in human pulmonary EC and incubated with the nitrating agent SIN-1, followed by immunoprecipitation with anti-Flag antibodies and detection with anti-nitrotyrosine antibodies. FIG. 4A shows results indicating that over-expressed S1PR3 exhibited basal tyrosine nitration that was substantially increased by SIN-1 challenge, findings consistent with S1PR3 receptor as a target for modification by tyrosine nitration.

### Example 4

### Barrier-Disruptive Agonists Induced S1PR3 Tyrosine Nitration and Release from EC

The capacity for the EC barrier-disrupting agent, lipopolysaccharide (LPS), to induce tyrosine nitration and alterations in cellular localization of the S1PR3 receptor were next investigated. LPS challenge (24 hrs) resulted in EC release of S1PR3 into the cellular media with extracellular S1PR3 exhibiting modification by tyrosine nitration (FIG. 4C) in human lung pulmonary artery EC (FIG. 4B). Nitrated S1PR3 levels in human lung microvascular EC were detected by both anti-nitrotyrosine and anti-S1PR3 immunoprecipitation and gel electrophoresis, further revealing the capacity for LPS to significantly increase nitrated S1PR3 in EC (24 hrs) (FIG. 4D).

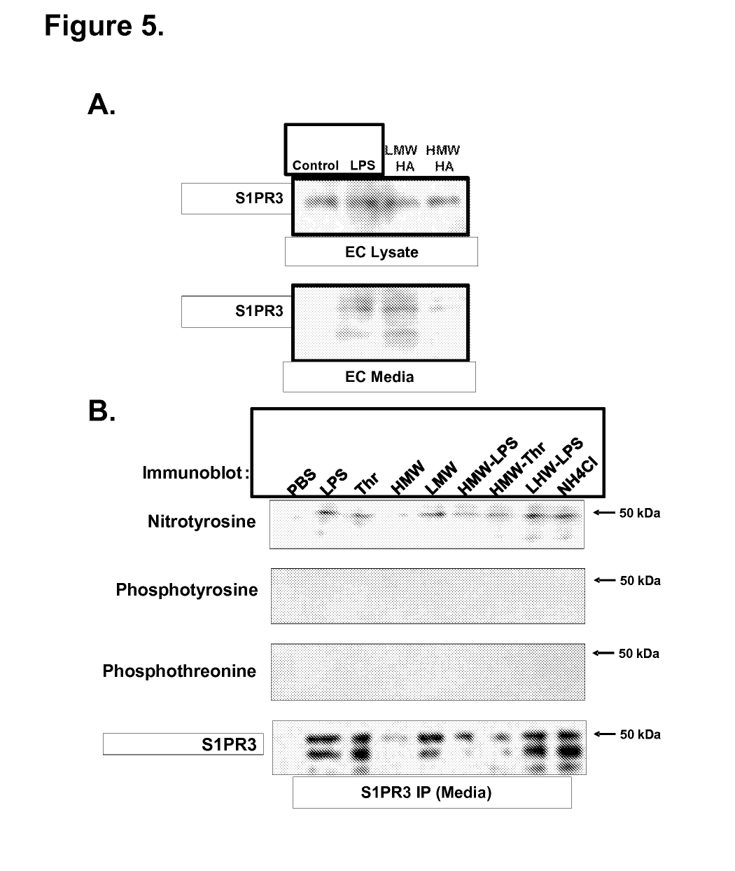

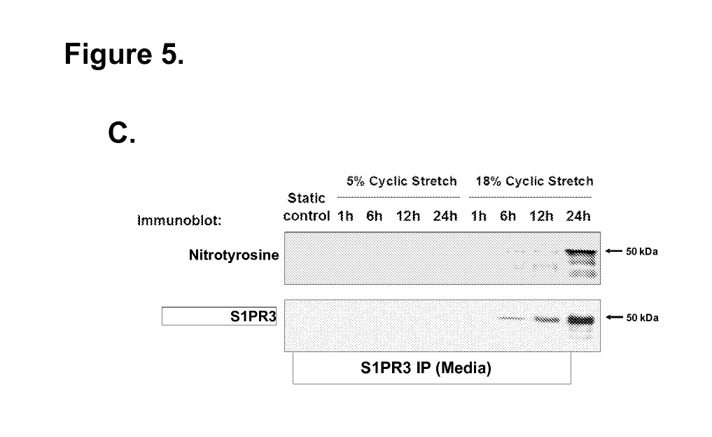

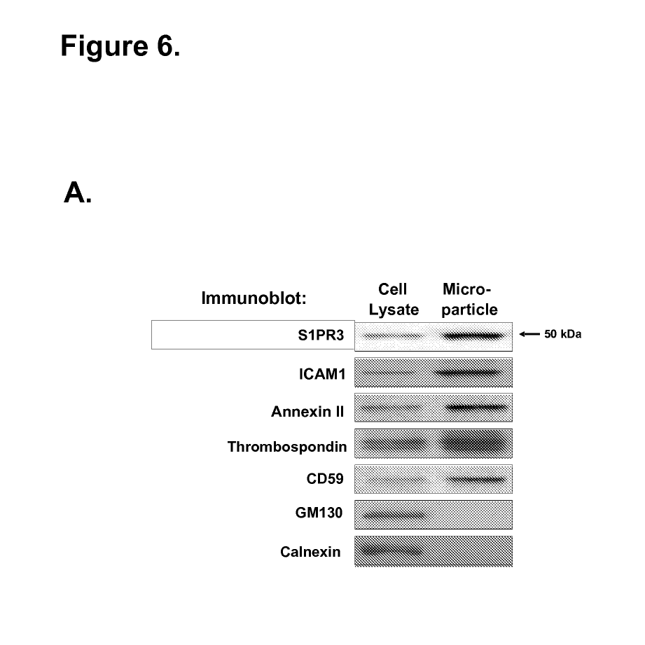

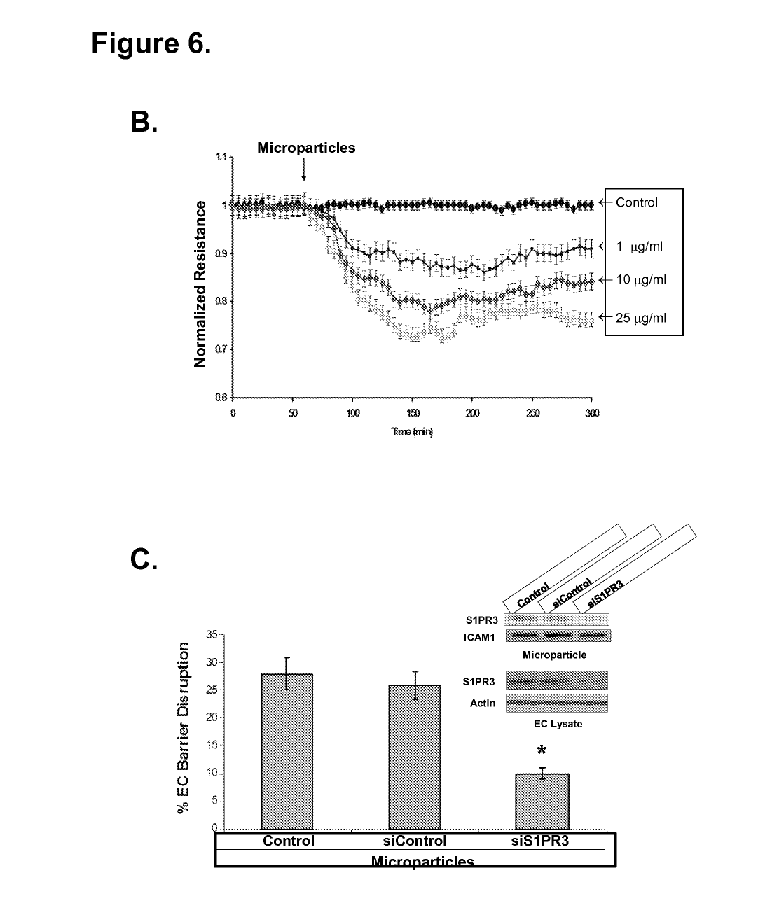

Whether additional vascular barrier-disruptive or barrier-enhancing agents regulate S1PR3 release into the media was also examined. In addition to LPS, the results shown in FIG. 5A confirmed S1PR3 release after the barrier-disrupting agent low molecular weight hyaluronan (LMW-HA) (Singleton, et al., J Biol Chem 2006; 281:34381-34393) (24 hrs) whereas the EC barrier-enhancing agent high molecular weight hyaluronan (HMW-HA) (Singleton, et al., 2006, J Biol Chem 2006; 281:34381-34393) failed to induce S1PR3 release. Consistent with regulation of S1PR3 release by EC barrier-disruptive agents, the results shown in FIG. 5B indicates that multiple barrier-disruptive agents LPS, thrombin, LMW-HA and ammonium chloride induced S1PR3 tyrosine nitration and cellular release with these responses attenuated by HMW-HA. However, in contrast to S1PR3 nitration, the results shown in FIG. 5B indicated that S1PR3 failed to exhibit tyrosine and threonine phosphorylation, post-translational modifications of S1PR3 previously demonstrated following LPS and thrombin (Singleton et al., Am J Respir Cell Mol Biol (2007) 37:222-31). Finally, as excessive mechanical stress is a well-recognized stimulus for lung inflammation and loss of vascular integrity (Hong et al., Am J Respir Crit Care Med (2008) 178:605-17), the effects of excessive mechanical stress via cyclic stretch were examined on EC release of S1PR3. Pathological barrier-disruptive cyclic stretch (18%) (Birukov et al., Am J Physiol Lung Cell Mol Physiol (2003) 285:L785-797; Ye et al., Am J Respir Crit Care Med (2005) 171:361-70; Nonas et al., Crit Care (2008) 12:R27) but not non-pathological, barrier-preserving cyclic stretch (5%), induced S1PR3 shedding and tyrosine nitration (6 to 24 hours) (as shown in FIG. 5C).

### Example 5

### Microparticles Containing Nitrated S1PR3 Enhanced EC Barrier-Disruption In Vitro

To examine the mechanism by which the S1PR3 is released from EC following exposure to barrier-disrupting agents, 10 to 1000 nm microparticles released from LPS-challenged EC containing intact plasma membrane proteins were isolated. Using differential centrifugation of LPS-challenged EC media (24 hr), materials were isolated containing known microparticle markers (ICAM-1, annexin II, thrombospondin, CD59) and excluding non-microparticle markers (GM130, calnexin) (FIG. 5A). LPS-induced microparticles contained significant S1PR3 content, suggesting a potential mechanism for cellular release from plasma membrane. As microparticles have been reported to cause vascular barrier disruption (Martin et al., Circulation (2004) 109:1653-59), the effects of microparticles isolated from LPS-challenged EC were examined on EC barrier integrity (normalized electrical resistance) and assessed whether S1PR3 in microparticles contributed to this process. The results shown in FIG. 5B indicated that the addition of isolated microparticles produce EC barrier disruption in a concentration-dependent manner. Microparticles derived from LPS-stimulated EC previously challenged with S1PR3 siRNAs to reduce S1PR3 expression, displayed a markedly reduced capacity to induce EC permeability compared to microparticles from EC exposed to silenced controls (as shown in FIG. 5C), indicating an important role for S1PR3 in microparticle-induced EC barrier disruption.

It should be understood that the foregoing disclosure emphasizes certain specific embodiments of the invention and that all modifications or alternatives equivalent thereto are within the spirit and scope of the invention as set forth in the appended claims.

I claim:

1. A method of determining risk of mortality from Acute Lung Injury (ALI), sepsis, or a combination thereof in a patient, the method comprising:
(a) obtaining a blood plasma sample from the patient; and
(b) performing an assay to measure tyrosine-nitrated sphingosine 1-phosphate 3 receptor (S1P3R) protein concentration in the plasma sample taken from the patient,
wherein there is an increased risk of mortality when said S1P3R protein concentration is 200 pg/mL or higher.

2. The method of claim 1, where there is an increased risk of mortality when the tyrosine-nitrated S1PR3 concentration is 250 pg/mL or higher.

3. The method of claim 1, wherein the tyrosine-nitrated S1P3R protein is isolated from a vascular endothelial cell-derived microparticle in said patient blood plasma sample.

4. The method of claim 1, wherein the tyrosine-nitrated S1P3R protein concentration is measured by an antibody-based detection method.

5. The method of claim 4, wherein the tyrosine-nitrated S1P3R protein concentration is measured by ELISA.

6. The method of claim 4, wherein the tyrosine-nitrated S1P3R protein is measured by western blot.

7. The method of claim 1, wherein the risk of mortality is increased by 10%, 20%, 30%, 40%, or 50%.

8. A method of diagnosing ALI in a patient with sepsis, the steps comprising:
(a) obtaining a blood plasma sample from the patient;
(b) performing an assay to measure tyrosine-nitrated S1P3R protein concentration in the plasma sample taken from the patient,
(c) performing an assay to measure tyrosine-nitrated S1P3R protein concentration in the plasma sample taken from a control patient, and
(d) comparing the protein concentrations determined in steps (b) and (c);
wherein ALI is diagnosed when S1P3R protein concentration is higher in the plasma sample determined in step (b) than in the plasma sample determined in (c).

9. The method of claim 7, wherein the tyrosine-nitrated S1P3R protein is isolated from a vascular endothelial cell-derived microparticle in said patient blood plasma sample.

10. The method of claim 8, wherein the tyrosine-nitrated S1P3R protein concentration is measured by an antibody-based detection method.

11. The method of claim 10, wherein S1P3R protein concentrations are measured by ELISA.

12. The method of claim 11, wherein S1P3 protein concentrations are measured by western blot.

13. A method of treating an Acute Lung Injury (ALI) patient with sepsis, the method comprising administering aggressive ALI treatment to the patient when S1P3R concentrations in the patient's plasma are at least 200 pg/mL.

14. The method of claim 13, wherein aggressive ALI treatment is given to the patient when S1P3R concentrations in the patient's plasma are at least 250 pg/mL.
